(19) United States
(12) Patent Application Publication
(10) Pub. No.: US 2025/0166784 A1
(43) Pub. Date: May 22, 2025

(54) COORDINATED CONTROL OF THERAPEUTIC TREATMENT EFFECTS

(71) Applicant: Cilag GmbH International, Zug (CH)

(72) Inventors: Frederick E. Shelton, IV, Hillsboro, OH (US); Chad E. Eckert, Terrace Park, OH (US); James T. Spivey, Whitehouse Station, NJ (US); Kevin M. Fiebig, Cincinnati, OH (US); Jason L. Harris, Lebanon, OH (US)

(21) Appl. No.: 18/810,266

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. G16H 20/40; G16H 10/60; G16H 50/20

(52) U.S. Cl. CPC G16H 20/40; G16H 10/60; G16H 50/20

(57) ABSTRACT

Devices, systems, and techniques for coordinated control of therapeutic treatment effects. An example device may select a therapeutic treatment from a database of a plurality of therapeutic treatments. The device may select a corresponding medical data stream relevant to the therapeutic treatment. The medical data stream may be produced by a surgical monitoring device. The device may determine, based on the medical data stream, qualitative feedback information about the therapeutic treatment.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

During an operation, a patient may receive therapeutic treatment. The treatment may have unintended primary and collateral effects on the patient's body. Without a method to monitor these effects, a surgeon may not be able to take remedial action in a timely manner.

Feature(s) described herein relate to controlling the boundaries and/or limitations of treatment systems to mitigate or prevent such unintended effects. For example, a first system may be providing a therapeutic treatment, and a second system may be monitoring the effects of the treatment. The controlled interaction between the two systems may allow a surgeon to adjust the treatment (e.g., the location or magnitude of the treatment) if needed.

A user may systematically coordinate the position and/or magnitude of the applied therapeutic treatment to control the shape of the primary and collateral effects of the treatment. For example, the user may control the location and/or operational parameter(s) of the therapy modality to define a three-dimensional therapeutic envelope of primary effect and secondary collateral interaction. Balancing the positional control and therapy effect may allow the user to control the primary and collateral envelope zones. An example intended primary effect may be a control percentage of cell death in a primary treatment zone. An example intended collateral effect may be an intended amount of cellular damage (e.g., but not cellular death) in a collateral treatment zone. The size and/or shape of the relational envelopes of the primary and collateral effects may be adjusted as needed.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example surgical operating room with therapy monitoring.

FIG. 6 illustrates an example therapy monitoring tool.

FIG. 7 illustrates example components of a therapy monitoring device.

FIG. 8 illustrates example selection options provided by the therapy monitoring device.

FIG. 9A illustrates and example therapy device inserted in bronchial tubes.

FIG. 9B illustrates the example therapy device of 9A providing cryoablation treatment to a tumor.

FIG. 9C illustrates therapy monitoring associated with the example therapy device of FIGS. 9A and 9B.

FIG. 10A illustrates an example therapy device providing chemotherapy treatment to a tumor.

FIG. 10B illustrates the therapy device of FIG. 10A identifying a chemotherapy leakage.

FIG. 10C illustrates the therapy device of FIGS. 10A and 10B adjusting the chemotherapy treatment in response to the identified leakage.

FIG. 10D illustrates therapy monitoring associated with the example therapy device of FIGS. 10A-C.

FIG. 11 illustrates an example method that may be performed by a therapy monitoring tool.

### DETAILED DESCRIPTION

A more detailed understanding may be had from the following description, given by way of example in conjunction with the accompanying drawings.

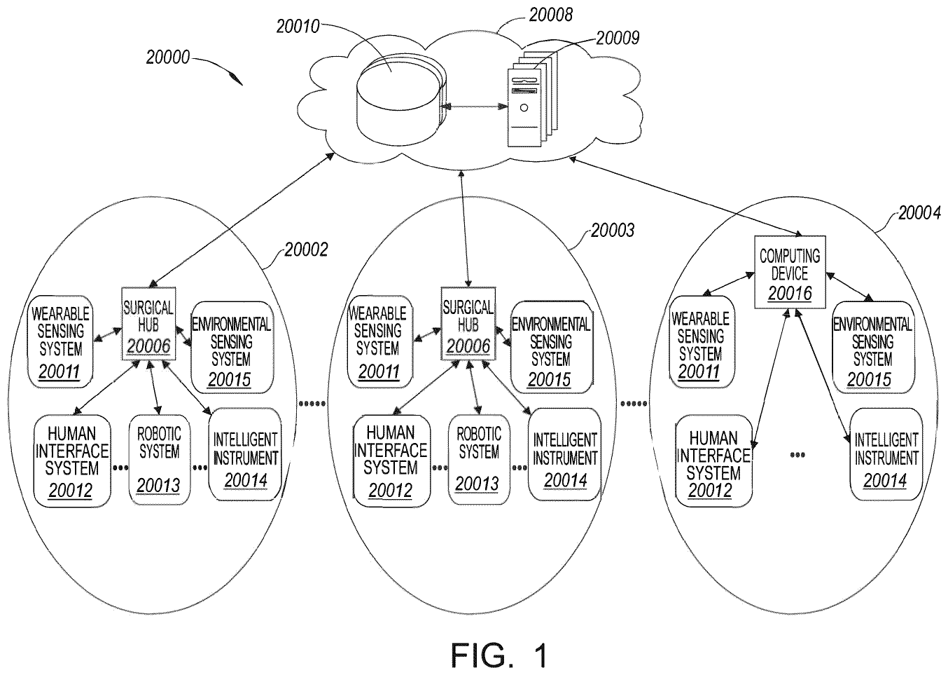

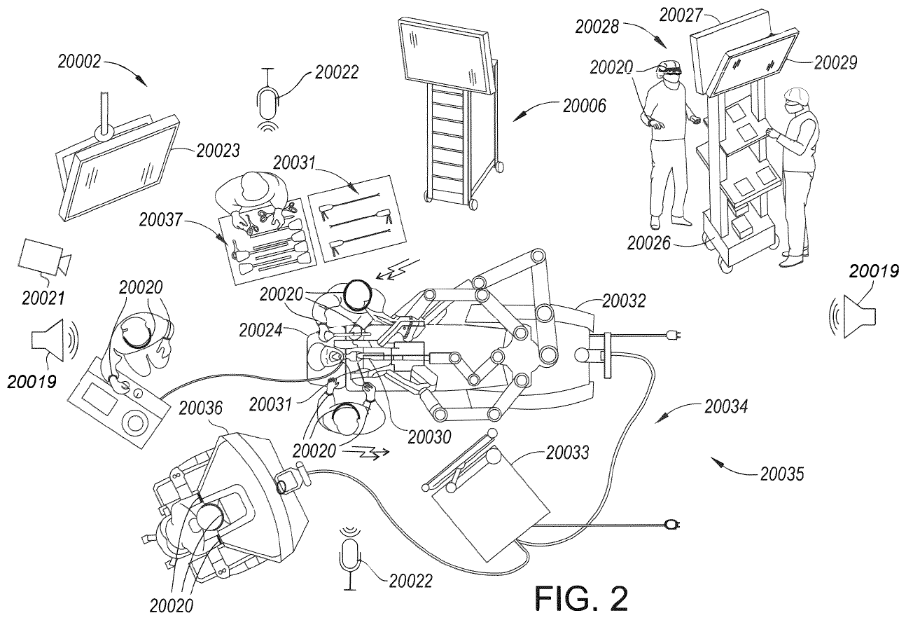

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

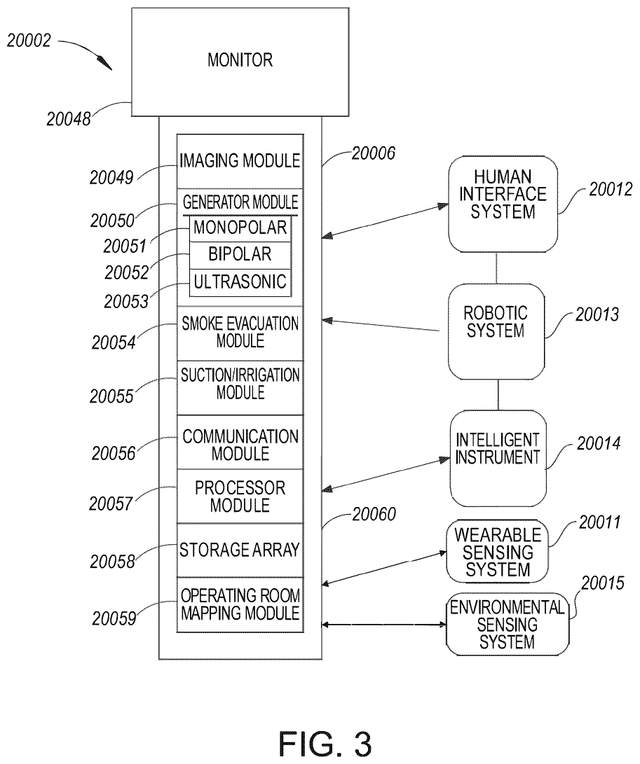

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

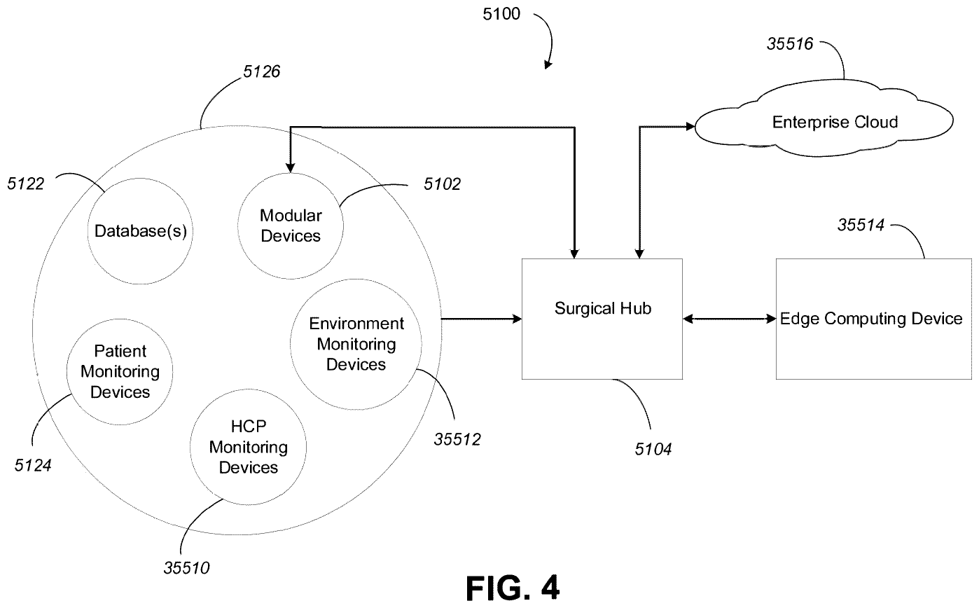

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

During an operation, a patient may receive therapeutic treatment. The treatment may have unintended primary and/or collateral effects on the patient's body. Without a method to monitor these effects, a surgeon may not be able to remedial action in a timely manner.

Feature(s) described herein relate to controlling the boundaries and/or limitations of treatment systems to mitigate or prevent such unintended effects. Systematic coordination may be accomplished using positional and magnitude control of the applied therapeutic treatment (e.g., to control the shape of the primary and collateral effects of the therapy).

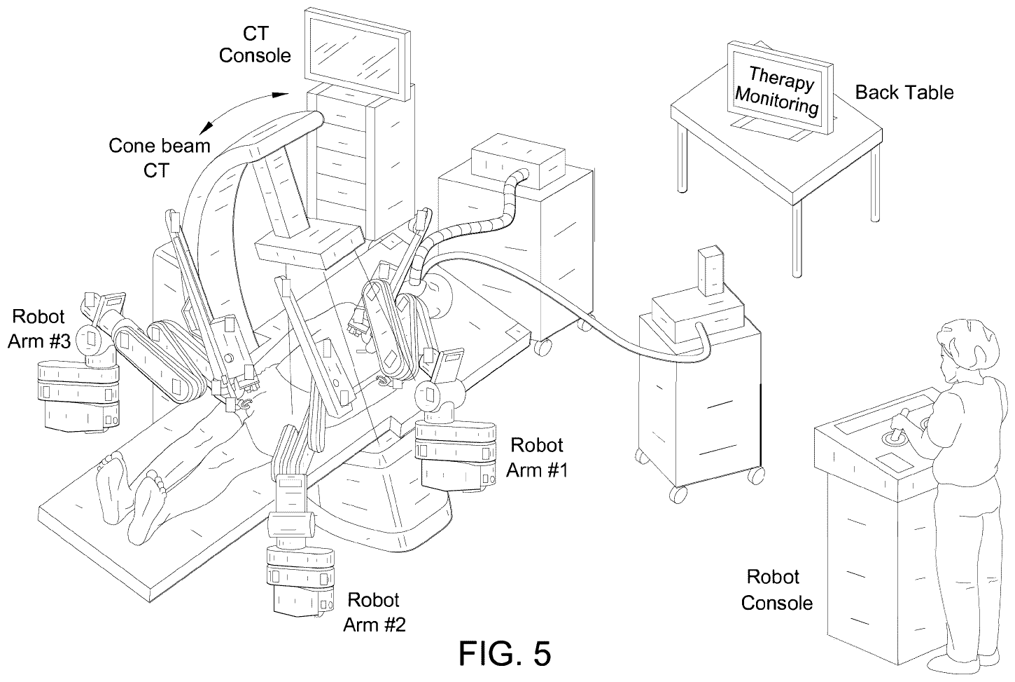

The location and/or operational parameter(s) of the therapy modality may be controlled to define a three-dimensional therapeutic envelope shape of primary effect and/or secondary collateral interaction. A surgical system may adjust one or more parameter(s) of the therapeutic treatment in response to the qualitative feedback information (e.g., information related to the effectiveness of the treatment). Example parameters of the therapeutic treatment may include one of more of: a location of the therapeutic treatment; a drug concentration; an injection pressure; a power level of a surgical instrument associated with the therapeutic treatment; a temperature associated with the surgical instrument; or a resolution of one or more imaging scans. FIG. 5 illustrates an example surgical operating room with therapy monitoring.

Systematic coordinated control of two related but independent operations may be used to control the boundaries of treatments. The interaction between systems may be controlled around an intended zone of effect. For example, one system may be providing therapy and the other system may be providing feedback control monitoring of the therapy or outcome.

The outcome of the treatment may be monitored (e.g., by a system that is independent of the system inducing the outcome). The monitoring may be used to control the magnitude of the effect applied. The monitored feed from a first smart system may be used to determine the magnitude of the effect of a second smart system. The treatment may be adjusted based on monitoring the outcome (e.g., by an independent system).

The primary therapy effect may be different from an intended collateral effect. The positional control may be balanced with the therapy applied effect to control the primary and collateral envelope zones. The intended primary effect may be a control percentage of cell death. The intended collateral effect may be an intended amount of cellular damage but not cellular death. The relational envelopes of the primary and collateral effects may be defined in terms of size and shape.

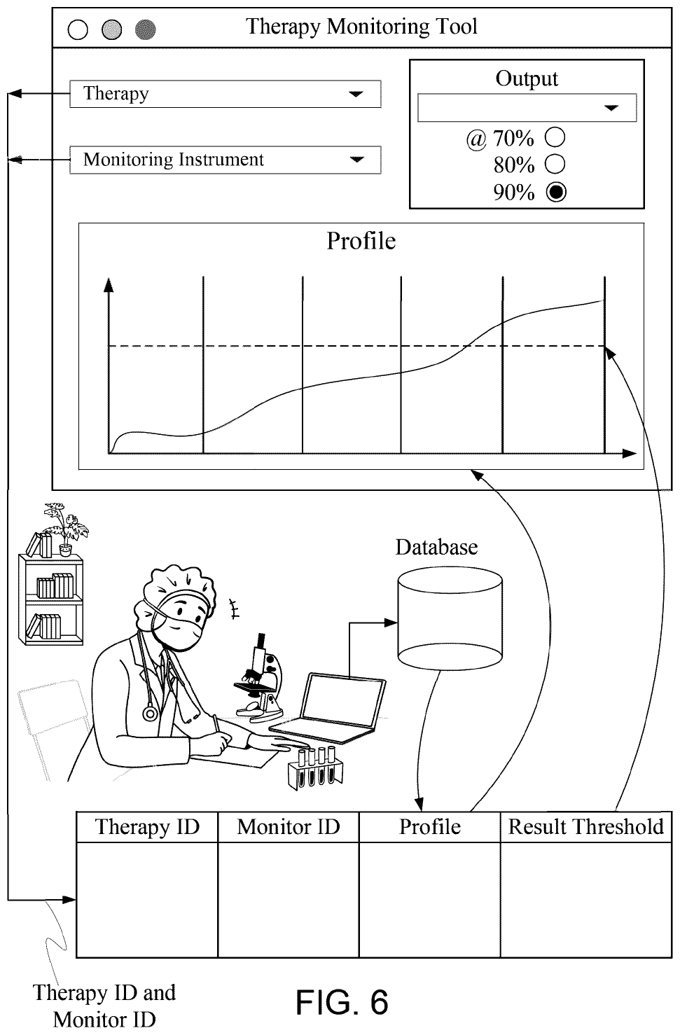

FIG. 6 illustrates an example therapy monitoring tool. The therapy monitoring tool may be an application (e.g., on a laptop or tablet) and/or incorporated in a surgical system (e.g., a surgical hub). The therapy monitoring tool may include a graphical user interface (GUI). An example GUI is illustrated at the top of FIG. 6. A user (e.g., medical professional such as a surgeon) may select (e.g., from drop down menus) a therapeutic treatment (e.g., a therapy type) and/or a corresponding medical data stream relevant to the therapeutic treatment (e.g., from a monitoring instrument). The monitoring instrument may be the surgical instrument that is capable of monitoring the level of therapy and/or the patient's response to the therapy (e.g., increased air inhalation, reduced heart rate, etc.). The user may select an output type. The user may select a threshold (e.g., 70%, 80%, or 90% in the example of FIG. 6). The threshold may indicate a threshold level of therapy to be applied, a threshold level of effect (e.g., biological response) of the treatment, and/or the like.

The user inputs may be used to generate a treatment profile. For example, the inputs may be sent to a database, and a profile matching the therapy ID and Monitor ID input by the user may be output. The therapy monitoring tool may display a graphical representation of the treatment profile. The graphical representation may include an indication (e.g., demarcation) of the threshold that was selected (e.g., as a dotted line showing the threshold). The graphical representation may include a graph showing the progress (and/or projected progress) of the treatment and/or the effect on the patient over time. For example, the graph may show a chemotherapy saturation level of a tumor over time. In this case, the user may be able to monitor the saturation level and cease treatment once the saturation level reaches 90%.

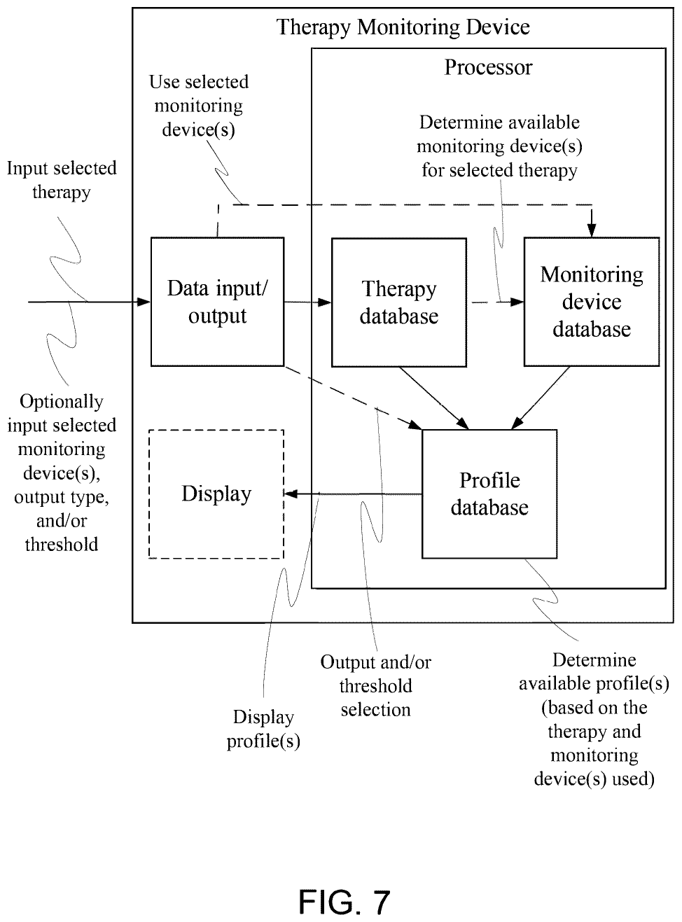

FIG. 7 illustrates example components of a therapy monitoring device. The therapy monitoring device may include a processor with one or more databases, a data input/output module, and/or a display. The data input/output module may receive user input indicating a selected therapeutic treatment. The user input may indicate a selected medical data stream from selected monitoring device(s) (e.g., or the therapy monitoring tool may select a suitable monitoring device based on the selected therapy).

The user input(s) may be funneled to respective database(s) (e.g., the selected therapy may be sent to a therapy database and the selected monitoring device may be sent to a monitoring device database). If a monitoring device is not selected, the therapy database may send an indication to the monitoring device database of an appropriate monitoring device to select. Based on the therapy and monitoring device(s) used, a profile database may determine available profile(s) that may be used to monitor the therapy. If the user selected an output type and/or threshold, the profile database may determine the available profile(s) based on that input as well. The profile(s) may be displayed (e.g., on a display of the therapy monitoring tool, and/or on a separate display).

The system may determine, based on the medical data stream and the selected therapeutic treatment, qualitative feedback information about the therapeutic treatment. The qualitative feedback may be indicative of an effectiveness of the therapeutic treatment. Determining the qualitative feedback information about the therapeutic treatment may involve determining an expected effect on a patient receiving the therapeutic treatment. The expected effect may be compared to the medical data stream. The qualitative feedback information may be determined based on the comparison.

Determining the qualitative feedback information about the therapeutic treatment may involve determining a threshold value associated with the medical data stream and the therapeutic treatment. The threshold value may be compared to the medical data stream. On a condition that the medical data exceeds the threshold value, the therapeutic treatment may be adjusted.

In an example, airflow inhalation and exhalation (tidal) volumes may be used as treatment indicators. Respiratory mechanics may refer to the interaction between the elastic/spongiform lungs, the elastic/rigid chest wall (thorax), and the bellows-like action of the diaphragm. During normal breathing, the diaphragm's excursion causes the lung to expand. The expansion creates a negative pressure (e.g., relative to normal air pressure) within the bronchial tree (e.g., 10-15 cm H₂O) with a resulting in-flow of room air at normal pressure. As the elastic recoil of the thorax and diaphragm occurs, the recently-inhaled gas escapes under slightly greater-than-room air pressure. This gas exchange allows the organism to ventilate (e.g., remove CO₂) and oxygenate. Two forms of mechanical ventilation are pressure ventilation (e.g., peak airway pressure constant, tidal volume variable) and volume ventilation (e.g., tidal volume constant, peak airway pressure variable.

In an example, the therapeutic treatment may be a tumor debulking therapy. The medical data stream may be a tidal volume of airflow at a given pressure. The qualitative feedback may be an indication of whether the tidal volume of airflow has reached a desired amount at the given pressure.

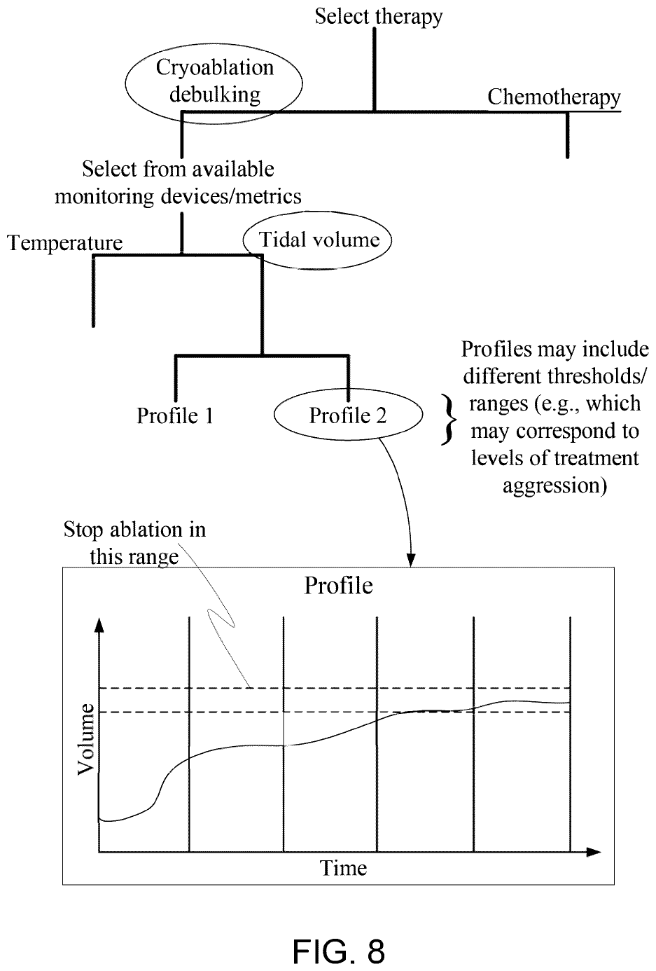

FIG. 8 illustrates example selection options provided by the therapy monitoring device. The user may select a therapy, in this example, cryoablation debulking. The user may then select a metric to monitor and/or a device to use for monitoring the metric. In this example, the user may select tidal volume as the metric. The therapy monitoring system may present two available profiles based on the user selections. For example, the profiles may have different thresholds and/or ranges (e.g., which may correspond to levels of treatment aggression). The therapy monitoring tool may display a graphical representation of the selected profile (e.g., Profile 2). The graphical representation may include a line illustrating when to stop ablation (e.g., a range of tidal volume that indicates that the tumor has been adequately debulked).

In this example, a smart ventilator may monitor the tidal volume of airflow at a given pressure. The smart ventilator may monitor the pressure at a given volume to monitor debulking therapy of a tumor. The debulking treatment may be used to reinstate airflow to a restricted portion of the patient's airways. The resumption of the amount of desired airflow at the desired pressure may be used as feedback to control the ablation system (e.g., to indicate that the tumor size has been sufficiently reduced). Tumor debulking may increase the chance that chemotherapy or radiation therapy will kill all the tumor cells. Tumor debulking may relieve symptoms or help the patient live longer.

Approximately one-third of patients with lung cancer will develop an airway obstruction. Many cancers lead to airway obstruction through metastases. Malignant Airway Obstruction (MAO) is when a tumor impinges on the bronchus connecting the main airway to the sub-sections of the lung. The treatment of MAO may involve a multi-modality approach. The treatment may be performed for palliation of symptoms in advanced lung cancer. Removal of an airway obstruction may be used to improve symptoms, quality of life, and lung function. Patients with short life expectancy, limited symptoms, and/or an inability to visualize beyond the obstruction may not be selected as candidates for this treatment.

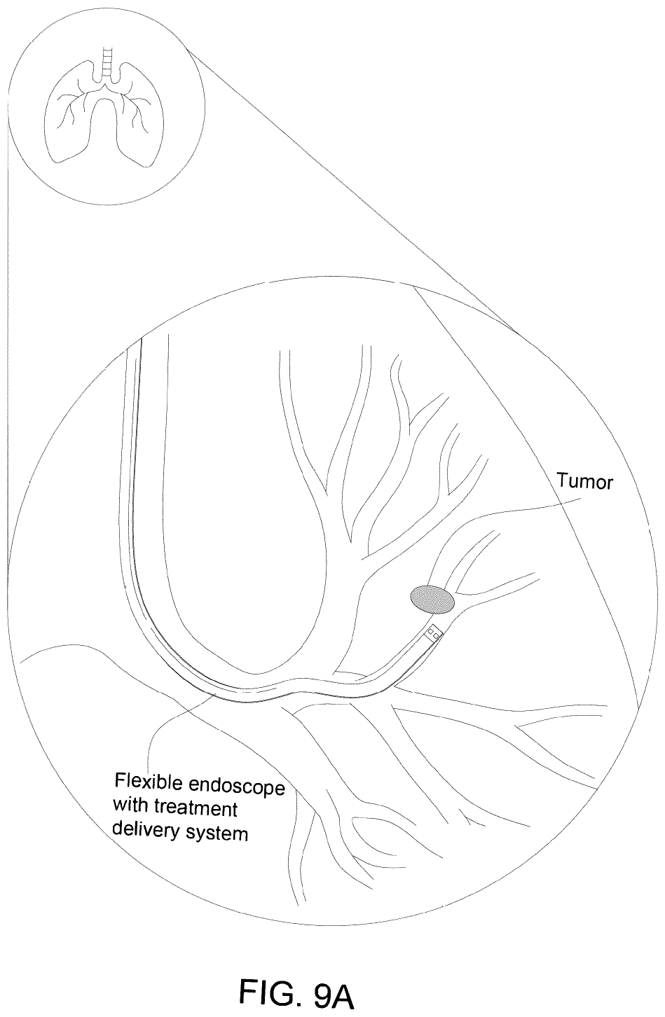

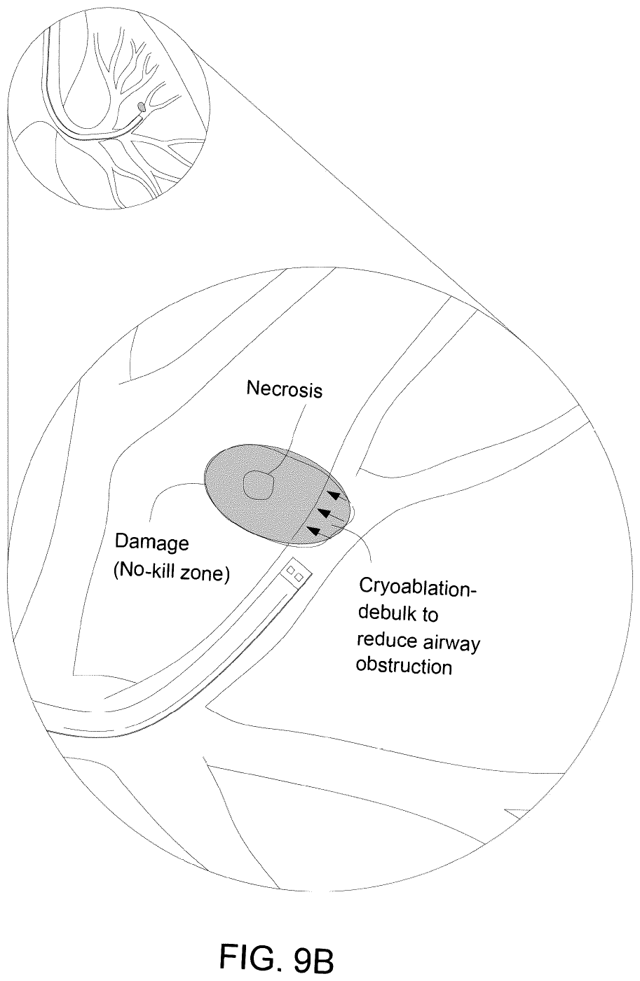

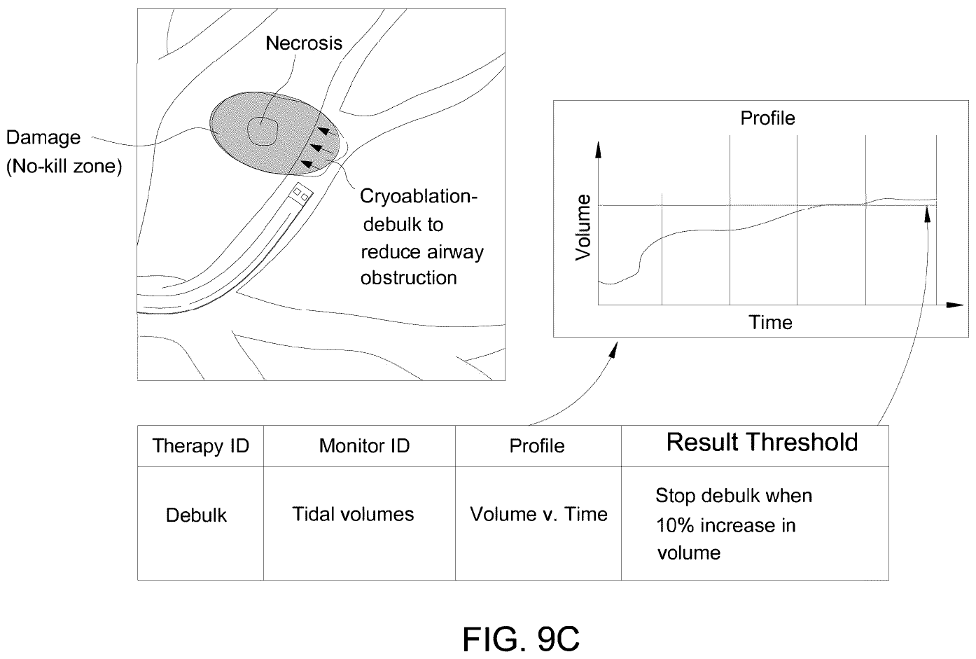

FIG. 9A illustrates and example therapy device inserted in bronchial tubes. In this example, a bronchial segment (e.g., the bronchus to segment three of the right lung) may have obstructed airflow (e.g., because of a tumor). This may reduce the tidal volume of breathing by about 10% from full capacity. As shown in FIG. 9A, a flexible endoscope may be used to enter the bronchial segment. The flexible endoscope may have a treatment delivery system (e.g., a needle with which to inject a drug, an ablation tool, and/or the like). An endoscopic robotic bronchoscopy procedure may be performed with a cryoablation, radio frequency monopolar ablation, or irreversible electroporation to reduce the size of a tumor and reestablish air exchange and/or reduce the tumor size for other tumor treatments (e.g., radiation therapy, chemo-therapy, immuno-therapy).

As shown in FIG. 9B, a portion of the tumor may be fully killed (e.g., necrotized). The remaining portion of the tissue may be damaged (but not killed) so that the body's immune response engage the remaining cancerous material. The killed portion of tissue may be exposed to sufficient levels of heat, cold, or electrical field to cause the destruction of the cells. Collateral interactions with adjacent cancerous cells may be limited in temperature, cold, or potential to leave the cells alive (e.g., but damage the cells to prevent undesired destruction of proteins that help the immune system target the remaining cells). The damaged cells may direct the immune system to the area to destroy the remaining cells (e.g., some proteins that initiate an immune response may not be destroyed). This treatment may cause the tumor to lose its hiding ability to the immune system so the immune system can remove and remodel the tissue.

The location of the fully-killed cells and the damaged cells may be controlled (e.g., based on the temperature used and the duration of the ablation and/or the magnitude of the patient's immune response). Heat or cold ablation may be used. Examples of the temperature ranges for heat ablation include temperatures greater than 60° C. to kill cells, and 35 to 45° C. to damage cells. Examples of the temperature ranges for cold ablation (e.g., cryoablation) include temperatures less than −30° C. to kill cells, and 0 to −10° C. to damage cells (e.g., a full therapeutic range for cryoablation is −30° C. to −75° C.).

The duration and temperature of the ablation may be used as control parameters for treatment. For example, a time-at-temperature transform may be used to determine cell death. For example, the therapeutic treatment may be an ablation therapy configured to produce a threshold amount of cell death. The medical data stream may be an ablation temperature and a time duration for which the ablation therapy has been applied. The qualitative feedback may include an indication of whether a time-at-temperature transform indicates that the threshold amount of cell death has been attained.

If a greater effect is desired, the time and/or temperature may be increased. The temperature ranges may be monitored by multi-spectral imaging to provide the data to the ablation system. The data from the multi-spectral imaging may be used as a predictive model of time and/or probe temperature (e.g., with no real-time real-tissue feedback on their effects on the temperature profile).

A portion of the tumor may be debulked (e.g., reduced in size to restore the air path allowing the segment three right to be functionally used). Debulking may be done using cryoablation. FIG. 9B illustrates the example therapy device of 9A providing cryoablation treatment to the tumor. In cryoablation, an extremely cold liquid (or an instrument called a cryoprobe) is used to freeze and destroy abnormal tissue. A cryoprobe may be cooled with substances such as liquid nitrogen, liquid nitrous oxide, or compressed argon gas. Cryoablation may refer to destroying tissue by freezing. Cryoablation may be used, for example, in the treatment of cancer. Cryoablation may shrink cancer tumor(s). Cryoablation may result in a significant decrease in pain (e.g., compared to pre-debulking and to other treatment methods). Cryoablation may be used to treat malignancy in a wide variety of organs (e.g., the eye, brain, head/neck, and esophagus). Cryoablation may be used in the treatment of liver, kidney, lung, prostate, and breast malignancy. Cryoablation may be performed via surgical (e.g., open or laparoscopic) or percutaneous approaches. Improvements in imaging (e.g., which allow earlier detection of smaller cancers) and a trend toward minimally invasive techniques in oncology have made image-guided oncologic intervention like cryoablation an attractive alternative to surgical treatments.

Cryoablation may be used to reduce the portion that is collapsing the bronchus (e.g., establishing two necrosis areas). A robotic flexible endoscope may use cryoablation to reduce the obstruction by 75%. A portion (e.g., 45%) of the tumor may be killed (e.g., by fully killing some cells). The endoscope may be used to control the position of the cryoablation. A closed control loop may use multi-spectral imaging (e.g., from the laparoscopic or endoscopic side) to monitor local temperatures within the kill, damage, and surrounding tissue zones. The control loop may balance the magnitude, pressure, and/or direction of the liquid (e.g., argon, nitrogen, or carbon dioxide) to keep the three zones within the desired ranges (e.g., and minimize bleed over from one zone to the next).

One or more parameters may be monitored by a smart system. For example, the system may monitor the location of the ablation device, the ablation temperature, power level, tissue impendence, tissue temperature, adjacent tissue temperature or thermal gradient, airway flow volume, volume (e.g., visualized 3D volume) of the tumor, and/or the extent of the acute ablative tumor debulking. FIG. 9C illustrates therapy monitoring associated with the example therapy device of FIGS. 9A and 9B.

A surgeon may determine that the debulking is sufficient if the surgeon sees a visualized size reduction of the impingement. The surgeon may monitor the tidal volume of the inhalation air flow as an indicator of the debulking sufficiency. For example, as illustrated in FIG. 9C, the surgeon may debulk the tumor and monitor the tidal volume until there is at least a 10% increase in volume at a set pressure. This technique may allow the debulking to be based on a size of the tumor reduction that was blocking the airway (e.g., rather than a fixed amount of tumor).

Examples of tumor obstructions of airways are provided herein. The airway obstructing tumor may affect an entire lung or lobe. Transbronchial tumors may be used to diagnose diffuse parenchymal lung disease (DPLD). The reported diagnostic yield may be between 70% to 80%. Tracheobronchial tumors may form in the inside lining of the trachea or bronchi (e.g., large airways of the lung). These may be a small portion of the overall Transbronchial tumors.

The patient's breathing may be used to control the de-bulking magnitude of the tumor located in the airway. Waveform capnography may indicate the amount of carbon dioxide (CO₂) in exhaled air (e.g., which may be used to assess ventilation). Waveform capnography may be depicted as a number and a graph. The number may represent the capnometry (e.g., the partial pressure of CO₂ detected at the end of exhalation). Transcutaneous oximetry (tcpO₂) may be used to measure the local oxygen released from the capillaries through the skin (e.g., reflecting the metabolic state of the lower limbs). tcpO₂ may be useful for wound healing prediction and qualification for hyperbaric oxygen therapy. tcpO₂ may be used to determine wound healing potential for patients undergoing amputation e.g., when used in conjunction with other clinical assessments).

The operational closed loop control features of device may be aggregated by a smart system. Some of the features may originate from the system itself. Some of the features may originate from another system external to the main control system. Data from the internal closed loop control and external critical operation features may be aggregated.

Local drug delivery may control cell growth or cell death. For example, the therapeutic treatment may involve injecting a chemotherapy (e.g., locally at a tumor). The medical data stream may be an imaging data stream of an area intended to receive the chemotherapy. The qualitative feedback may include a first indication of whether the chemotherapy has leaked outside of the area intended to receive the chemotherapy. The qualitative feedback may include a second indication of whether the area intended to receive the chemotherapy has reached a threshold level of chemotherapy saturation.

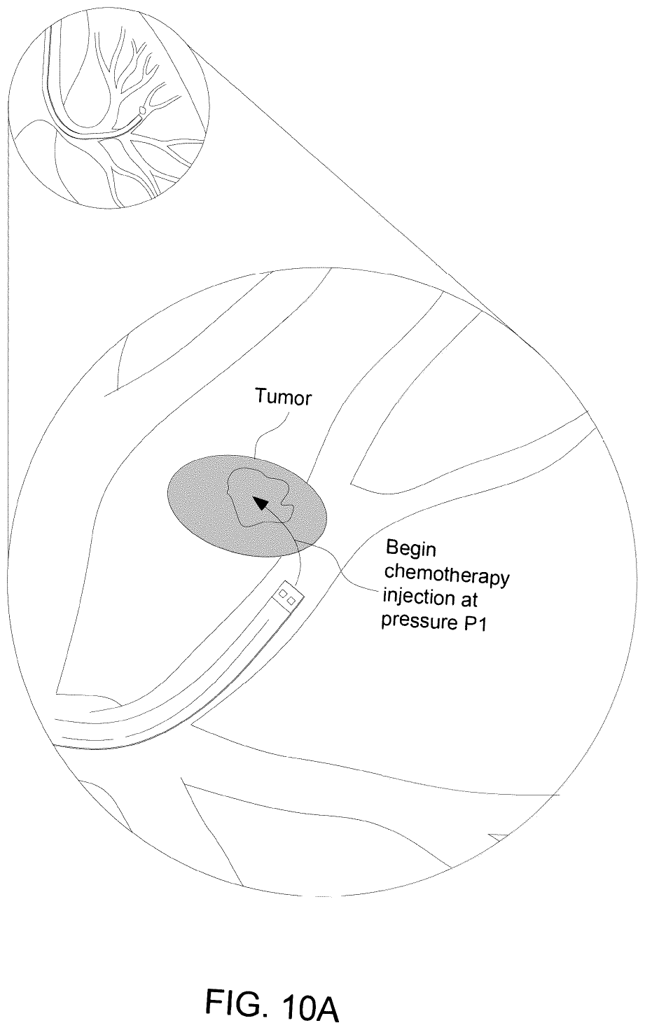

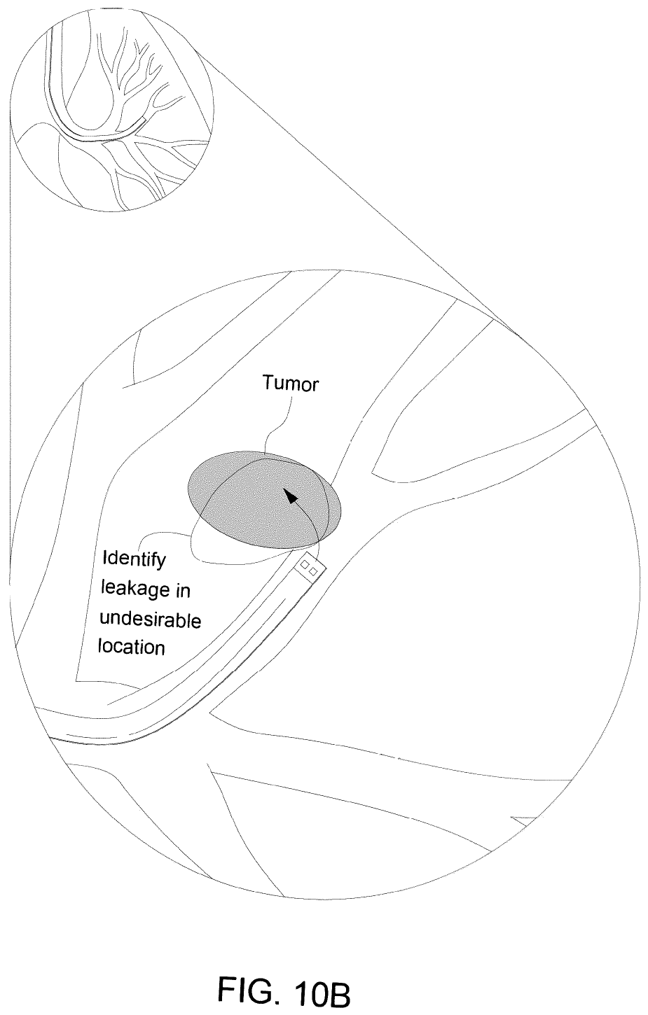

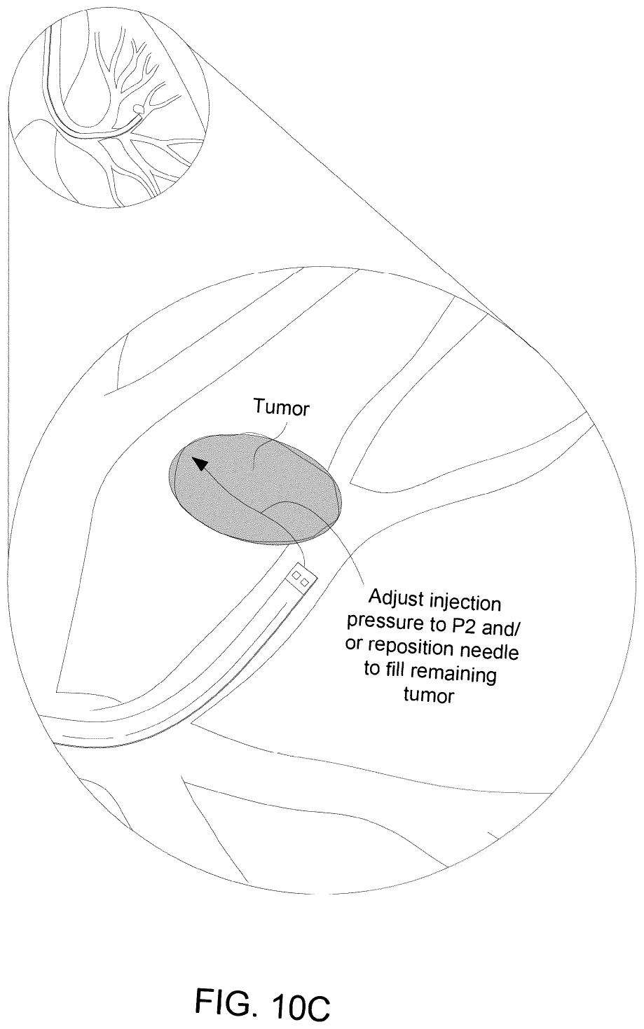

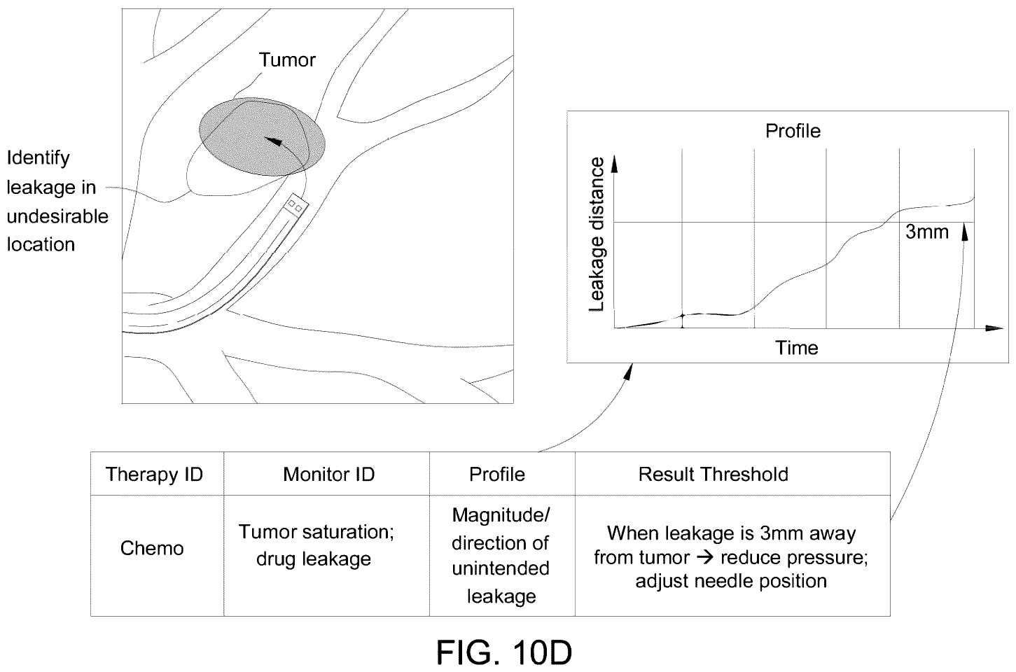

FIG. 10A illustrates an example therapy device providing chemotherapy treatment to a tumor. In this example, the lung cancer tumor occurs mid-parenchyma. The tumor may not be spherical and/or have irregular nodules. The robotic flexible endoscope may approach the tumor from the bronchus side. The endoscope may insert the treatment needle to a position close to center of the tumor (e.g., by using the cone beam CT or ultrasound guidance).

In an example, a flexible endoscopic robot (e.g., used for a bronchoscopy) may have a biopsy needle. The endoscopic robot may deliver drugs locally to a tumor (e.g., for treatment at the time and site of biopsy). The endoscopic robot may be guided to the tumor through the bronchi. The needle may extend through the working channel to biopsy the tumor. A surgeon may determine a method and magnitude of local drug delivery. The system may determine the needle position, needle angle, needle depth, and drug fluid injection pressure. As illustrated in FIG. 10A, the endoscopic robot may begin administering a drug (in this example, chemotherapy). The endoscopic robot may begin injecting the drug at a first pressure P1.

A smart monitoring system may control the needle insertion depth, needle angle relative to the tumor, positioning of the needle within the tumor, drug fluid injection pressure, drug dosage, drug volume, and/or other parameters. The parameters may be monitored through advanced visualization systems (e.g., multi-spectral, cone beam CT, Ultrasound, etc.). These parameters may be monitored to control the overall saturation of the desired area of the tumor with the desired dosage of the local drug delivery. FIG. 10B illustrates the therapy device of FIG. 10A identifying a chemotherapy leakage.

An independent system may monitor control of the treatment dispersal system. Radio opaque drug monitoring may be used to identify leaks (e.g., magnitude and direction of leaks) and/or saturation/completeness of tumor treatment. CT scanning may be used to monitor the drug saturation and/or concentration of the treatment area(s). Multi-spectral imaging may provide the margin sizes and effects to a cone beam CT (e.g., based on vascularity, reflectivity, and fluorescence). Local margin boundary detection may be used. An endoscope may output navigation information (e.g., triangulation coordinates) for the cone beam CT machine to use in focusing and positioning.

The injection pressure of the drug may be monitored to ensure full saturation of the planned tumor area. Irregular mass ejections outside of the tumor (e.g., in undesired locations, which may result in tissue death in unintended areas) may be monitored. The drug concentration may therefore be controlled in the intended areas and accidentally exposed areas.

For example, the endoscope may inject a contrast-doped medicant to fully saturate the tumor (e.g., with up to a concentration of 20%). The medicant may bloom out from the needle location in a spherical manner. As the medicant approaches the first edge of the tumor, it may squirt out of the tumor through a crevasse into the adjacent non-tumor tissue. The cone-beam CT may identify the leak and change the injection pressure of the fluid.

The initial injection pressure level (e.g., P1 in FIG. 10A) may be high. As illustrated in FIG. 10B, a leak may form, causing the chemotherapy drug to escape into unintended/undesirable location(s). FIG. 10C illustrates the therapy device of FIGS. 10A and 10B adjusting the chemotherapy treatment in response to the identified leakage. As shown, the system may reduce the injection pressure to a second pressure P2 (e.g., to reduce additional leakage). The system may change the position and/or orientation of the needle to one or more other location(s) to inject towards the untreated portion of the tumor (e.g., to fill the rest of the tumor to the minimum desired concentration of the chemotherapy drug) and minimize additional leakage. The adjustments may continue until the tumor (e.g., all of the tumor) is treated (e.g., with between 20-30% concentration of the mendicant).

A first limiting threshold may indicate the maximum amount of leakage or exposure of tissue outside of the tumor. The first limiting threshold my include a margin that is acceptable before the application is stopped and/or adjusted. A second limiting threshold may indicate the minimum concentration of the drug within the tumor and within the margin. The minimum concentration may apply to the surface area of the desired extent of the tumor treatment (e.g., shape, size, concentration, and/or the like).

FIG. 10D illustrates therapy monitoring associated with the example therapy device of FIGS. 10A-C. In this example, the system may notify the surgeon when the leakage is 3 mm or more away from the edge of the tumor. The surgeon may use this indication as a cue to reduce the injection pressure and/or adjust the needle position. As another example, a drug concentration threshold may be used to indicate the end of the procedure. For example, if the system detects that 90% or more of the tumor has a threshold concentration of the drug, the surgeon may conclude the treatment. In these examples, if the position and/or pressure adjustments were not made, a significant amount of the medicant may have spread beyond the tumor. Similarly, a significant amount of the tumor may be under-treated if the adjustments were not made.

Situational control of smart system boundaries may be based on independent (e.g., unrelated) events. For example, if a chemotherapy drug has just been applied to the patient in a specific area, cooling of the patient in that area should be avoided (e.g., so that maximum effectiveness of drug delivery is obtained). If a drug is delivered to the patient while the patient temperature is in a cooled or cooling state, drug absorption may be impacted (e.g., reduced or delayed). In this case, upon preparing for drug delivery or post drug delivery, a boundary of where cooling therapies are applied may be reduced.

Multiple (e.g., two) systems that have cooperated to achieve a balance between them may be adjusted (e.g., for a discrete portion of time) to induce an imbalance that effects the tissue or surgical space (e.g., to improve a specific step of a surgical procedure).

For example, in an endoscopic mucosal resection (EMR) (e.g., as the energy on a hot snare from an endoscopic-colon robot increases), endoscopic insufflation pressure may decrease and laparoscopic pressure may increase. This may reduce resistance while snaring the lesion to be resected (e.g., as the snare cuts). Robotic control of snare deployment (e.g., out of the working instrument distal tube) may trigger the synchronized change of the laparoscopic pressure and endoscopic pressure.

In an example, the therapeutic treatment may involve designing an implant for a patient. The medical data stream may include a first imaging scan of an anatomical area of a patient associated with the implant and a second imaging scan of the anatomical area of the patient associated with the implant. The qualitative feedback may include an indication of whether inconsistencies between the first and second imaging scans exist or whether anomalies exist in either the first or second imaging scans.

For example, CT scans may be used to create precisely adapted patient-specific implants. The CT scans of the patient may be taken. The data from the scans may be transferred to a system or outside company (e.g., 3Di). The system or outside company may process the data and create a virtual patient model. Once the virtual patient model has been confirmed, a 3D implant model may be created. The 3D implant model may be reviewed/confirmed by the doctor. The implant may be manufactured and sent to site for implant.

Multiple scan sources may be combined to further enhance the model created (e.g., rather than only utilizing a CT scan). For example, ultrasound and/or magnetic resonance imaging (MRI) may be used. The processing side may interact with the CT scanning system to identify inputs that do not generate correctly and/or anomalies identified during the virtual model. This interaction may minimize potential errors or differences within a patient to create a more accurate representation of the implant.

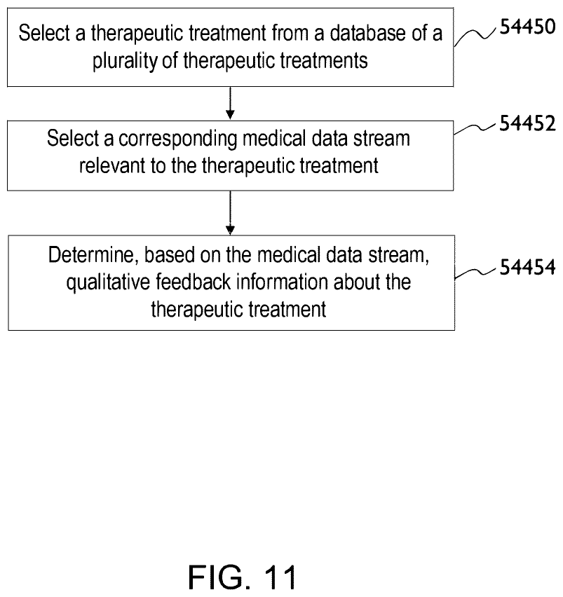

FIG. 11 illustrates an example method that may be performed by a therapy monitoring tool. As shown at 54450, the method may involve selecting a therapeutic treatment from a database of a plurality of therapeutic treatments. The method may involve, at 54452, selecting a corresponding medical data stream relevant to the therapeutic treatment, wherein the medical data stream is produced by a surgical monitoring device. The method may involve, at 54454, determining, based on the medical data stream, qualitative feedback information about the therapeutic treatment.

What is claimed:

1. A device comprising:
a user input interface;
a display;
a first interface with a surgical device to send control information indicative of a therapeutic treatment;
a second interface to receive, from a surgical monitoring device, a medical data stream with information indicative of operation of the surgical device; and
a processor configured to:
receive, via the user input interface, a therapeutic treatment selected by a user;
retrieve, from a therapeutic treatment database, therapeutic treatment information associated with the selected therapeutic treatment;
select the surgical monitoring device, from a monitoring device database, based on the therapeutic treatment information, wherein the surgical monitoring device monitors medical data, included in the medical data stream, relevant to efficacy of the therapeutic treatment;
generate a therapy profile based on the therapeutic treatment information and the surgical monitoring device, wherein the therapy profile comprises a graphical representation of progress of the therapeutic treatment;
output the therapy profile to the display;
receive, over the first interface, the medical data stream;
determine, based on the medical data, qualitative feedback information about the therapeutic treatment;
determine a threshold value associated with the medical data and the therapeutic treatment;
compare the medical data to the threshold value determined from the therapy profile; and
on a condition that the medical data exceeds the threshold value, adjust, over the first interface, the therapeutic treatment.

2. The device of claim 1, wherein the therapeutic treatment comprises a tumor debulking therapy, the medical data stream comprises a tidal volume of airflow at a given pressure, and the qualitative feedback comprises an indication of whether the tidal volume of airflow has reached a desired amount at the given pressure.

3. The device of claim 1, wherein the therapeutic treatment comprises injecting a chemotherapy, the medical data stream comprises an imaging data stream of an area intended to receive the chemotherapy, and the qualitative feedback comprises a first indication of whether the chemotherapy has leaked outside of the area intended to receive the chemotherapy, and a second indication of whether the area intended to receive the chemotherapy has reached a threshold level of chemotherapy saturation.

4. The device of claim 1, wherein the therapeutic treatment comprises an ablation therapy configured to produce a threshold amount of cell death, the medical data stream comprises an ablation temperature and a time duration for which the ablation therapy has been applied, and the qualitative feedback comprises an indication of whether a time-at-temperature transform indicates that the threshold amount of cell death has been attained.

5. A method comprising:
selecting a therapeutic treatment from a database of a plurality of therapeutic treatments;
selecting a corresponding medical data stream relevant to the therapeutic treatment, wherein the medical data stream is produced by a surgical monitoring device; and
determining, based on the medical data stream, qualitative feedback information about the therapeutic treatment.

6. The method of claim 5, wherein the qualitative feedback information about the therapeutic treatment is indicative of an effectiveness of the therapeutic treatment.

7. The method of claim 5, wherein determining, based on the medical data stream, qualitative feedback information about the therapeutic treatment comprises:
determining an expected effect on a patient receiving the therapeutic treatment;
comparing the medical data stream to the expected effect; and
determining the qualitative feedback information based on the comparison.

8. The method of claim 5, wherein determining, based on the medical data stream, qualitative feedback information about the therapeutic treatment comprises:
determining a threshold value associated with the medical data stream and the therapeutic treatment;
comparing the medical data to the threshold value; and
on a condition that the medical data exceeds the threshold value, adjusting the therapeutic treatment.

9. The method of claim 5, wherein the therapeutic treatment comprises a tumor debulking therapy, the medical data stream comprises a tidal volume of airflow at a given pressure, and the qualitative feedback comprises an indication of whether the tidal volume of airflow has reached a desired amount at the given pressure.

10. The method of claim 5, wherein the therapeutic treatment comprises injecting a chemotherapy, the medical data stream comprises an imaging data stream of an area intended to receive the chemotherapy, and the qualitative feedback comprises a first indication of whether the chemotherapy has leaked outside of the area intended to receive the chemotherapy, and a second indication of whether the area intended to receive the chemotherapy has reached a threshold level of chemotherapy saturation.

11. The method of claim 5, wherein the therapeutic treatment comprises an ablation therapy configured to produce a threshold amount of cell death, the medical data stream comprises an ablation temperature and a time duration for which the ablation therapy has been applied, and the qualitative feedback comprises an indication of whether a time-at-temperature transform indicates that the threshold amount of cell death has been attained.

12. The method of claim 5, wherein the therapeutic treatment comprises designing an implant, the medical data stream comprises a first imaging scan of an anatomical area of a patient associated with the implant and a second imaging scan of the anatomical area of the patient associated with the implant, and the qualitative feedback comprises an indication of whether inconsistencies between the first and second imaging scans exist or whether anomalies exist in either the first or second imaging scans.

13. The method of claim 5, wherein the method further comprises adjusting a parameter of the therapeutic treatment in response to the qualitative feedback information, and wherein the parameter of the therapeutic treatment comprises one of more of:
a location of the therapeutic treatment;
a drug concentration;
an injection pressure;
a power level of a surgical instrument associated with the therapeutic treatment;
a temperature associated with the surgical instrument; or
a resolution of one or more imaging scans.
